(19) United States
(12) Patent Application Publication
(10) Pub. No.: US 2025/0387176 A1
(43) Pub. Date: Dec. 25, 2025

(54) CALIBRATING A SURGICAL ROBOT ARM

(71) Applicant: CMR Surgical Limited, Cambridge (GB)

(72) Inventors: Dominic Martin McBrien, Cambridge (GB); Isaac Uden, Cambridge (GB)

(73) Assignee: CMR Surgical Limited, Cambridge, OT (GB)

(21) Appl. No.: 18/880,437

(22) PCT Filed: Jun. 12, 2023
(86) PCT No.: PCT/GB2023/051525

§ 371 Date: Dec. 31, 2024

(30) Foreign Application Priority Data
Jul. 1, 2022 (GB) 2209738.0

(51) Int. Cl. A61B 34/30; A61B 17/00; A61B 34/00; A61B 90/00

(52) U.S. Cl. CPC A61B 34/30; A61B 17/00; A61B 34/77; A61B 90/06; A61B 2017/00725; A61B 2034/305; A61B 2090/066

(57) ABSTRACT

A method for calibrating a surgical robot arm motor includes running a test sequence. The test sequence includes controlling a set of test motor currents to be applied to the motor, each causing the motor to drive a drive interface element to move; and for each test motor current, receiving a measured resistive force applied by a calibration rig; determining a relationship between the set of test motor currents and the resistive force measurements; determining calibration value(s) from: (i) the determined relationship, and (ii) a known relationship between the resistive force applied by the calibration rig and the driving force applied by the drive interface element; and controlling the calibration value(s) to be applied to subsequent motor currents applied to the motor so as to cause the motor to drive the drive interface element to apply desired driving forces to an instrument interface element of an attached surgical instrument.

### BACKGROUND

It is known to use robots for assisting and performing surgery. FIG. 1 illustrates a typical surgical robotic system. A surgical robot 100 consists of a base 102, an arm 104 and an instrument 106. The base supports the robot, and may itself be attached rigidly to, for example, the operating theatre floor, the operating theatre ceiling or a cart. The arm extends between the base and the instrument. The arm is articulated by means of multiple flexible joints 108 along its length, which are used to locate the surgical instrument in a desired location relative to the patient. The surgical instrument is attached to the distal end of the robot arm. The surgical instrument penetrates the body of the patient at a port so as to access the surgical site. The surgical instrument comprises a shaft connected to a distal end effector 110 by a jointed articulation. The end effector engages in a surgical procedure. In FIG. 1, the illustrated end effector is a pair of jaws.

A surgeon controls the surgical robot 100 via a remote surgeon console 112. The surgeon console comprises one or more surgeon input devices 114. These may take the form of a hand controller or foot pedal. The surgeon console also comprises a display 116.

A control system 118 connects the surgeon console 112 to the surgical robot 100. The control system receives inputs from the surgeon input device(s) 114 and converts these to control signals to move the joints of the robot arm 104 and instrument 106. The control system sends these control signals to the robot, where the corresponding joints are driven accordingly.

The surgical instrument is attached to the robot arm at an interface. Interface elements of the robot arm engage interface elements of the surgical instrument. Drive is transferred from the robot arm to the surgical instrument mechanically at the interface via the interface elements. Specifically, motors in the robot arm drive interface elements of the robot arm. Those interface elements of the robot arm are engaged with and hence transfer drive to the interface elements of the instrument. The interface elements of the instrument transfer this drive to the distal end effector via the internal structure of the instrument. For example, in a cable driven instrument, the force applied by the interface elements of the robot arm to the interface elements of the instrument is transferred to the cables which drive joints of the instrument's articulation to move the distal end effector.

Thus, when, for example, the surgeon input device 114 commands the jaws of surgical instrument 110 to close, the control system 118 responds by controlling the current applied to motors in the robot arm to drive interface elements of the robot arm to move. These drive interface elements transfer drive to interface elements of the instrument. Those instrument interface elements transfer drive to driving cables in the instrument which cause the jaws to rotate towards each other.

Calibration of the current applied to each motor of the robot arm is important to ensure that the instrument moves as commanded by the surgeon input device 114. Delivering a consistent force to the instrument is particularly important for gripping actions of an instrument which has opposable end effector elements, such as jaws or scissors.

### SUMMARY OF THE INVENTION

According to an aspect of the invention, there is provided a method for calibrating a motor of a surgical robot arm using a calibration rig, the motor configured to drive a drive interface element of the surgical robot arm, the drive interface element configured to drive an instrument interface element of a surgical instrument attached to the surgical robot arm to thereby drive a distal end effector of the surgical instrument, the calibration rig comprising a rig interface element which engages with and is driven by the drive interface element, the method comprising: running a test sequence comprising: controlling a set of test motor currents to be applied to the motor, each test motor current causing the motor to drive the drive interface element to move; and for each test motor current of the set of test motor currents, receiving a measured resistive force applied by a calibration rig, the calibration rig applying the resistive force in response to the rig interface element being driven by the drive interface element; determining a relationship between the set of test motor currents and the resistive force measurements; determining calibration value(s) from: (i) the determined relationship, and (ii) a known relationship between the resistive force applied by the calibration rig and the driving force applied by the drive interface element; and controlling the calibration value(s) to be applied to subsequent motor currents applied to the motor so as to cause the motor to drive the drive interface element to apply desired driving forces to an instrument interface element of an attached surgical instrument.

The method may further comprise, prior to running the test sequence, attaching the calibration rig to the surgical robot arm.

The method may further comprise, for each test motor current, measuring the resistive force applied by the calibration rig at the calibration rig.

The resistive force applied by the calibration rig may be proportional to the velocity of the drive interface element.

The determined relationship may be a linear relationship, the calibration value(s) being a factor and/or offset.

The resistive force applied by the calibration rig may be the same as the driving force applied by the drive interface element.

The method may comprise determining an average resistive force measurement of a plurality of resistive force measurements taken for each test motor current, and determining the relationship between the set of test motor currents and the average resistive force measurements.

The motor may drive the drive interface element in a linear direction.

The motor may drive the drive interface element to rotate.

The method may further comprise setting up the robot arm in a predetermined test configuration prior to running the test sequence.

The surgical robot arm may comprise a further motor for driving a further drive interface element, the further drive interface element being configured to drive a further instrument interface element of the surgical instrument, the method further comprising repeating the steps to calibrate the further motor.

The steps may be implemented for the motor and the further motor concurrently.

The surgical robot arm may comprise an arm force sensor configured to measure the driving force applied by the motor to the drive interface element. The method may further comprise:

for each test motor current of the set of test motor currents, measuring the driving force at the arm force sensor; determining a further relationship between the set of test motor currents and the driving force measurements; determining further calibration value(s) from the determined further relationship; and applying the calibration value(s) to the arm force sensor.

The determined further relationship may be a linear relationship, the calibration value(s) being a factor and/or offset.

The method may further comprise: driving the motor with a maximum current; whilst driving the motor with the maximum current: measuring the resistive force, and at the arm force sensor, measuring the driving force applied by the motor to the drive interface element; comparing the measured resistive force to a resistive force tolerance threshold; comparing the measured driving force to a driving force tolerance threshold; if either the measured resistive force does not meet the resistive force tolerance threshold, or the measured driving force does not meet the driving force tolerance threshold, performing the calibration method. The method may further comprise: comparing the measured resistive force to the driving force tolerance threshold; and if the measured resistive force does not meet the driving force tolerance threshold, performing the calibration method.

The method may further comprise: comparing the measured driving force to the resistive force tolerance threshold; and if the measured driving force does not meet the resistive force tolerance threshold, performing the calibration method.

The method may further comprise: comparing the measured resistive force to a predetermined force limit, and halting the test sequence if the measured resistive force exceeds the predetermined force limit.

The method may further comprise, for each test motor current of the set of test motor currents, measuring a constant velocity of the drive interface element.

The method may further comprise, for each test motor current of the set of test motor currents, measuring the distance travelled by the drive interface element whilst the drive interface element moves at a constant velocity.

The method may further comprise verifying the motor calibration by: applying a verification motor current to the motor to drive the drive interface element to move at a verification velocity; for that verification motor current, measuring the verification resistive force applied by the calibration rig; and comparing the measured verification resistive force to a target force.

The method may further comprise verifying the arm force sensor calibration by: for the verification motor current, measuring the verification driving force at the arm force sensor; and comparing the measured verification resistive force to the verification driving force.

According to a second aspect of the invention, there is provided a method of calibrating an arm force sensor of a surgical robot arm using a calibration rig, the arm force sensor configured to measure the driving force applied by a motor of the robot arm to a drive interface element of the robot arm, the drive interface element configured to drive an instrument interface element of a surgical instrument attached to the surgical robot arm to thereby drive a distal end effector of the surgical instrument, the calibration rig comprising a rig interface element which engages with and is driven by the drive interface element, the method comprising: running a test sequence comprising: controlling a set of test motor currents to be applied to the motor, each test motor current causing the motor to drive the drive interface element to move; and for each test motor current of the set of test motor currents, receiving (i) a measured resistive force applied by the calibration rig, the calibration rig applying the resistive force in response to the rig interface element being driven by the drive interface element, and (ii) a measured driving force at the arm force sensor; determining a relationship between the resistive force measurements and the driving force measurements; determining calibration value(s) from the determined relationship; and controlling the calibration value(s) to be applied to the arm force sensor.

According to a third aspect of the invention, there is provided a calibration rig configured to calibrate a motor of a surgical robot arm, the motor configured to drive a drive interface element of the surgical robot arm, the drive interface element configured to drive an instrument interface element of a surgical instrument attached to the surgical robot arm to thereby drive a distal end effector of the surgical instrument, the calibration rig comprising: a rig interface element shaped so as to engage with and be driven by the drive interface element; a damper configured to provide a resistive force in response to the rig interface element being driven by the drive interface element; and a rig force sensor configured to, for each of a set of test motor currents applied to the motor to drive the drive interface element to move, measure the resistive force applied by the damper.

The damper may be a linear damper configured to provide a resistive force proportional to the constant velocity of the driven drive interface element.

The linear damper may be configured to only provide the resistive force in one linear direction.

The linear damper may be configured to provide the resistive force in two opposing linear directions.

The rig force sensor may be a load cell in-line with the linear damper.

The calibration rig may further comprise a further rig interface element shaped so as to engage with and be drive by a further drive interface element.

The calibration rig may further comprise a further damper configured to provide a resistive force in response to the further rig interface element being driven by the further drive interface element.

The calibration rig may further comprise a further rig force sensor configured to measure the resistive force applied by the further damper.

### BRIEF DESCRIPTION OF THE FIGURES

The present invention will now be described by way of example with reference to the accompanying drawings. In the drawings:

FIG. 1 illustrates a surgical robot system for performing a surgical procedure;

FIG. 2 illustrates a surgical robot;

FIG. 3 illustrates an exemplary surgical instrument;

FIGS. 4a and 4b illustrate the distal end of an exemplary surgical instrument;

FIG. 5 illustrates a drive assembly interface;

FIG. 6 illustrates an instrument interface configured to engage the drive assembly interface of FIG. 5;

FIG. 7 illustrates a calibration rig;

FIGS. 8a, 8b and 8c illustrate alternative calibration rig configurations which connect to a drive assembly interface which provides a rotary drive;

FIG. 9 is a flowchart of a method of calibrating a motor of the robot arm which drives a drive interface element;

FIG. 10 is a schematic diagram of the apparatus used to carry out the method of FIG. 9;

FIG. 11 is a graph illustrating the resistive force measured at different drive interface element positions whilst a test motor current is applied to the motor;

FIG. 12 is a graph illustrating the resistive force against drive interface element position for a set of test motor currents applied to the motor;

FIG. 13 is a plot illustrating the relationship between motor force and measured resistive force;

FIG. 14 illustrates a drive assembly interface;

FIG. 15 is a flowchart of a method of calibrating an arm force sensor which drives a drive interface element; and

FIG. 16 is a flowchart of a method of determining whether to perform the calibration methods of FIGS. 9 and 15.

### DETAILED DESCRIPTION

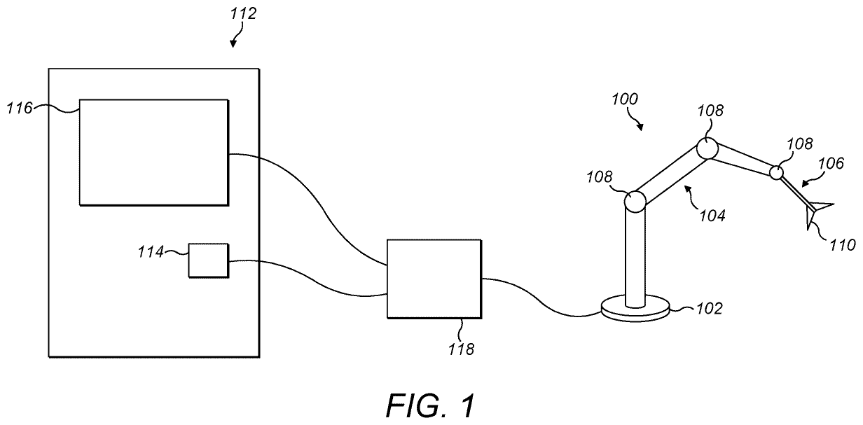

The following describes a calibration rig and a method of utilising it to calibrate motor currents of a surgical robot arm. The calibration rig may also be used to calibrate force sensors of the surgical robot arm. The calibration rig attaches to the surgical robot arm at an interface. Usefully, that interface is the same interface at which the surgical instrument attaches to the surgical robot arm. The surgical robotic arm and surgical instrument form part of a surgical robotic system of the type illustrated in FIG. 1.

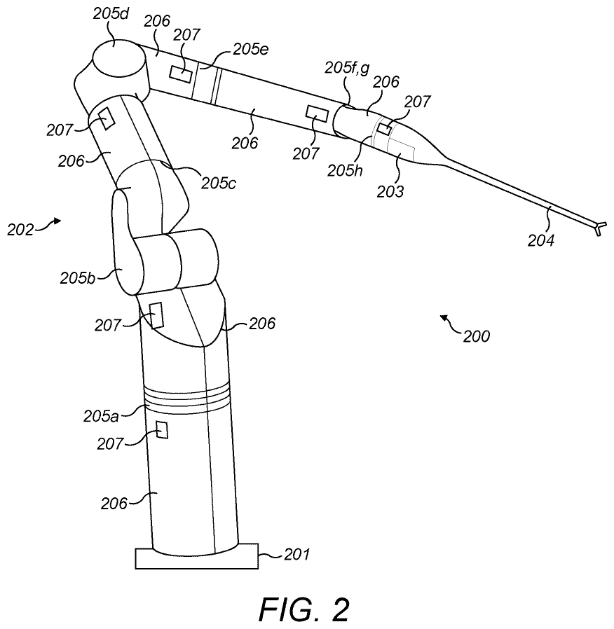

FIG. 2 illustrates an example robot 200. The robot comprises a base 201 which is fixed in place when a surgical procedure is being performed. Suitably, the base 201 is mounted to a chassis. That chassis may be a cart, for example a bedside cart for mounting the robot at bed height. Alternatively, the chassis may be a ceiling mounted device, or a bed mounted device.

A robot arm 202 extends from the base 201 of the robot to a terminal link 203 to which a surgical instrument 204 can be attached. The arm is flexible. It is articulated by means of multiple flexible joints 205 along its length. In between the joints are rigid arm links 206. The arm in FIG. 2 has eight joints. The joints include one or more roll joints (which have an axis of rotation along the longitudinal direction of the arm members on either side of the joint), one or more pitch joints (which have an axis of rotation transverse to the longitudinal direction of the preceding arm member), and one or more yaw joints (which also have an axis of rotation transverse to the longitudinal direction of the preceding arm member and also transverse to the rotation axis of a co-located pitch joint). In the example of FIG. 2: joints 205a, 205c, 205e and 205h are roll joints; joints 205b, 205d and 205f are pitch joints; and joint 205g is a yaw joint. Pitch joint 205f and yaw joint 205g have intersecting axes of rotation. The order of the joints from the base 201 to the terminal link 203 of the robot arm is thus: roll, pitch, roll, pitch, roll, pitch, yaw, roll. However, the arm could be jointed differently. For example, the arm may have fewer than eight or more than eight joints. The arm may include joints that permit motion other than rotation between respective sides of the joint, for example a telescopic joint. The robot comprises a set of drivers 207. Each driver 207 has a motor which drives one or more of the joints 205. The terminal link 203 of the robot arm comprises a drive assembly for interfacing and driving a surgical instrument. The drive assembly will be described in more detail below.

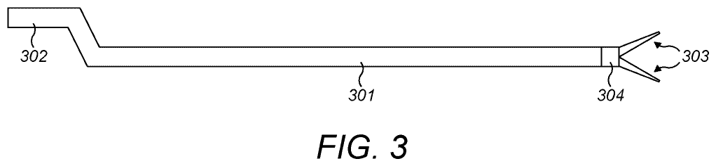

FIG. 3 illustrates a surgical instrument 204. The surgical instrument has an elongate profile, with a shaft 301 spanning between its proximal end which is attached to the robot arm and its distal end which accesses the surgical site within the patient body. Suitably, the shaft is rigid. The shaft may be straight. The proximal end of the surgical instrument and the instrument shaft may be rigid with respect to each other and rigid with respect to the distal end of the robot arm when attached to it. At the proximal end of the instrument, the shaft 301 is connected to an instrument interface 302. The instrument interface engages with the drive assembly interface at the distal end of the robot arm as will be described in more detail below. At the distal end of the surgical instrument, the distal end of the shaft is connected to an end effector 303 by an articulation 304. The end effector 303 engages in a surgical procedure at the surgical site. The end effector may take any suitable form. For example, the end effector could be a pair of curved scissors, an electrosurgical instrument such as a pair of monopolar scissors, a needle holder, a pair of jaws, or a fenestrated grasper.

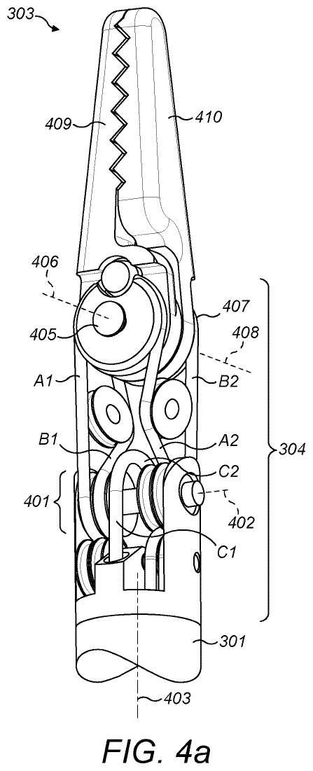

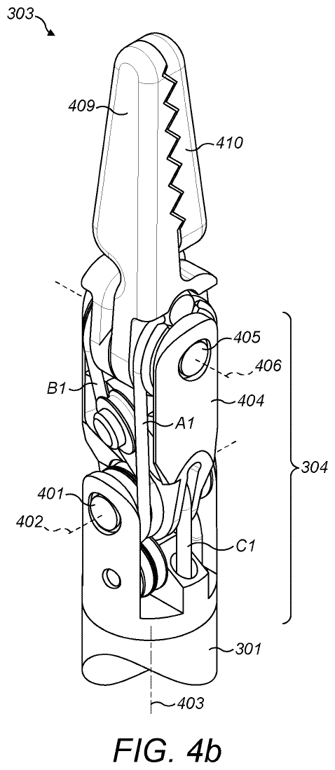

FIGS. 4a and 4b illustrate the distal end of an exemplary instrument which has a pair of jaws as the end effector 303. The shaft 301 is connected to the end effector 303 by articulation 304. The articulation 304 comprises several joints. These joints enable the pose of the end effector to be altered relative to the direction of the instrument shaft. Although not shown in FIGS. 4a and 4b, the end effector may also comprise joint(s). In the example of FIGS. 4a and 4b, the articulation 304 comprises a pitch joint 401. The pitch joint 401 rotates about pitch axis 402, which is perpendicular to the longitudinal axis 403 of the shaft 301. The pitch joint 401 permits a supporting body 404 (described below) and hence the end effector 303 to rotate about the pitch axis 402 relative to the shaft. In the example of FIGS. 4a and 4b, the articulation also comprises a first yaw joint 405 and a second yaw joint 407. First yaw joint 405 rotates about first yaw axis 406. Second yaw joint 407 rotates about second yaw axis 408. Both yaw axes 406 and 408 are perpendicular to pitch axis 402. Yaw axes 406 and 408 may be parallel. Yaw axes 406 and 408 may be collinear. The articulation 304 comprises a supporting body 404. At one end, the supporting body 404 is connected to the shaft 301 by pitch joint 401. At its other end, the supporting body 404 is connected to the end effector 303 by the yaw joints 405 and 407. This supporting body is omitted from FIG. 4a for ease of illustration so as to enable the other structure of the articulation to be more easily seen.

The end effector 303 shown comprises two end effector elements 409, 410. Alternatively, the end effector may have a single end effector element. The end effector elements 409, 410 shown in FIGS. 4a and 4b are opposing jaws. However, the end effector elements may be any type of opposing end effector elements. The first yaw joint 405 is fast with the first end effector element 409 and permits the first end effector element 409 to rotate about the first yaw axis 406 relative to the supporting body 404 and the pitch joint 401. The second yaw joint 407 is fast with the second end effector element 410 and permits the second end effector element 410 to rotate about the second yaw axis 408 relative to the supporting body 404 and the pitch joint 401. In FIGS. 4a and 4b, the end effector elements 409, 410 are shown in a closed configuration in which the jaws abut.

The joints illustrated in FIGS. 4a and 4b are driven by pairs of driving elements. These driving elements run through the shaft from the instrument interface to the articulation. The driving elements are elongate. They are flexible transverse to their longitudinal extent. They resist compression and tension forces along their longitudinal extent. A first pair of driving elements A1, A2 are constrained to move around the first yaw joint 405. Driving elements A1, A2 drive rotation of the first end effector element 409 about the first yaw axis 406. FIGS. 4a and 4b illustrate a second pair of driving elements B1, B2 which are constrained to move around the second yaw joint 407. Driving elements B1, B2 drive rotation of the second end effector element 410 about the second yaw axis 408. FIGS. 4a and 4b also illustrate a third pair of driving elements C1, C2 which are constrained to move around pitch joint 401. Driving elements C1, C2 drive rotation of the end effector 303 about the pitch axis 402. The end effector 303 can be rotated about the pitch axis 402 by applying tension to driving elements C1 and/or C2. The pitch joint 401 and yaw joints 405, 407 are independently driven by their respective driving elements. In the example of FIGS. 4a and 4b, there are three pairs of driving elements driving the joints of the articulation. In alternative examples, there may be only two pairs of driving elements driving the joints of the articulation.

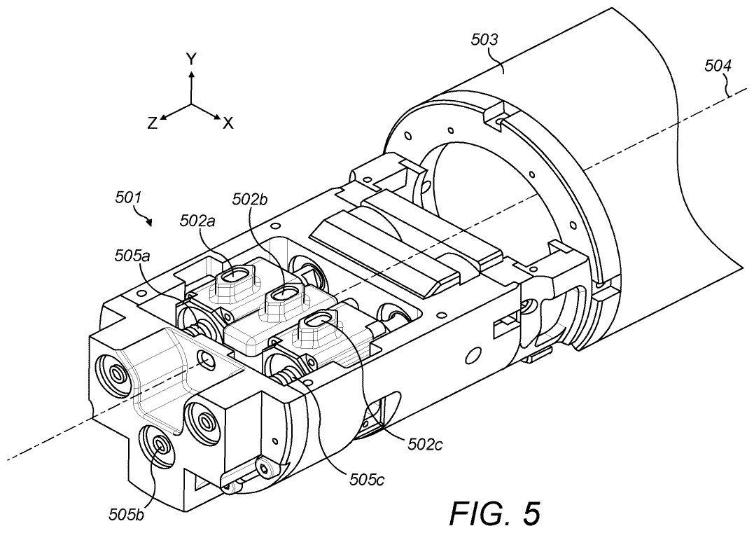

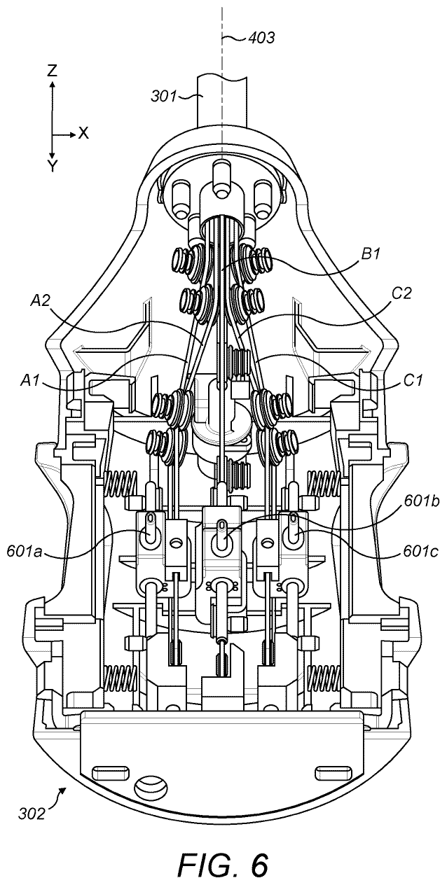

FIGS. 5 and 6 illustrate an exemplary engageable instrument interface 302 and drive assembly interface 501. The drive assembly interface, shown in FIG. 5, is attached to the terminal link 503 of the robot arm. The drive assembly interface 501 comprises a plurality of drive interface elements 502a, 502b, 502c. FIG. 5 illustrates three drive interface elements. These drive interface elements all lie in the same plane parallel to the longitudinal axis 504 of the terminal link of the robot arm. Each drive interface element is moveable within the drive assembly interface. Each drive interface element is displaceable parallel to the longitudinal axis of the terminal link of the robot arm. In this example, each drive interface element is driven along its range of motion by a lead screw 505a, 505b, 505c with which it is in threaded engagement. Each lead screw is in turn driven by a motor in the robot arm. Thus, each drive interface element moves parallel to the other drive interface elements in the same plane parallel to the longitudinal axis 504 of the terminal link of the robot arm.

FIG. 6 illustrates an instrument interface 302 configured to engage with the drive assembly interface of FIG. 5. The instrument interface 302 is attached to the shaft 301 of the instrument. The instrument interface 302 comprises a plurality of instrument interface elements 601a, 601b, 601c. FIG. 6 illustrates three instrument interface elements. Each instrument interface element is attached to a pair of the driving elements A1, A2, B1, B2, C1, C2. The instrument interface elements 601a, 601b, 601c all lie in the same plane parallel to the longitudinal axis 403 of the shaft 301 of the instrument. Each instrument interface element is moveable within the instrument interface. Each instrument interface element is displaceable parallel to the longitudinal axis of the instrument shaft, thereby displacing the attached driving element. Each instrument interface element moves parallel to the other instrument interface elements in the same plane parallel to the longitudinal axis 403 of the shaft 301 of the instrument.

A robotic surgical instrument having the instrument interface of FIG. 6 engages a surgical robot arm having the drive assembly interface of FIG. 5 in a direction Y perpendicular to the longitudinal axes of the terminal link 504 of the robot arm and the instrument shaft 403. When the robot arm and instrument are engaged, the longitudinal axis of the terminal link 504 of the robot arm is parallel to the longitudinal axis 403 of the instrument shaft. The longitudinal axis of the terminal link 504 of the robot arm may be aligned with the longitudinal axis 403 of the instrument shaft. When the robotic surgical instrument engages the surgical robot arm, instrument interface element 601a engages drive interface element 502a, instrument interface element 601b engages drive interface element 502b, and instrument interface element 601c engages drive interface element 502c. As each drive interface element moves relative to the drive assembly interface across its range of motion it transfers drive to the instrument interface element it is engaged with. That instrument interface element thus moves relative to the instrument interface across its range of motion thereby transferring drive to its attached pair of driving elements.

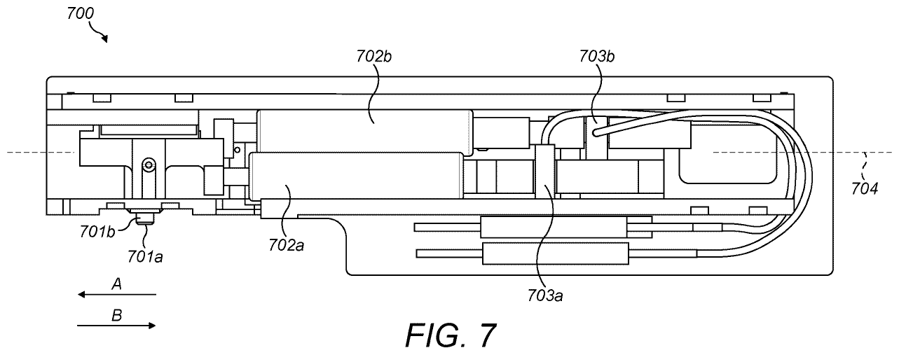

FIG. 7 illustrates a calibration rig 700 for calibrating a motor 207 of the robot arm which drives a drive interface element 502 of the robot arm. The calibration rig 700 attaches to the drive assembly of the robot arm in the same manner as the instrument interface of the instrument. The calibration rig 700 may comprise a latch mechanism to securely attach to the drive assembly of the robot arm. The calibration rig 700 comprises a rig interface element 701a for engaging a drive interface element 502a. The rig interface element 701a is shaped so as to engage with and be driven by the drive interface element 502a. Thus, the rig interface element 701a has a complimentary shape to the drive interface element 502a. Suitably, the rig interface element 701a has the same shape and size as instrument interface element 601a. The rig interface element 701a is displaceable in a linear direction. Suitably, this linear direction is parallel to the longitudinal axis 704 of the calibration rig. When the calibration rig 700 is attached to the robot arm, the rig interface element 701a moves linearly parallel to the longitudinal axis 504 of the end of the robot arm. The rig interface element 701a displaces over a displacement range which is the same as, or longer than, the displaceable range of the drive interface element 502a which it engages. Thus, the rig interface element 701a is driven by the drive interface element 502a in the same way as the instrument interface element 601a.

The calibration rig 700 comprises a damper 702a to which the rig interface element 701a is attached. The damper 702a provides a resistive force in response to the rig interface element 701a being driven by the drive interface element 502a. In the example of FIG. 7, the damper 702a is a linear damper. The resistive force provided by the linear damper 702a is proportional to the velocity at which the rig interface element 701a moves. Hence, the resistive force provided by the linear damper 702a is proportional to the velocity at which the drive interface element 502a moves. Any suitable linear damper 702a may be used. For example, the linear damper may be a hydraulic linear damper.

The calibration rig 700 of FIG. 7 also comprises a rig force sensor 703a. The rig force sensor measures the resistive force applied by the damper 702a. The rig force sensor 703a may, for example, be a load cell in-line with the linear damper. The rig force sensor 703a may be located on either side of the damper 702a. In FIG. 7, the damper 702a is between the rig interface element 701a and the rig force sensor 703a. However, alternatively, the rig force sensor 703a may be between the rig interface element 701a and the damper 702a.

Calibration rig 700 is most usefully used to calibrate the motors of the robot arm which drive opposable end effector elements of an attached surgical instrument to rotate. Opposable end effector elements 409 and 410 are driven to rotate by drive elements A1, A2 and B1, B2. Drive elements A1, A2 are driven by instrument interface element 601a. Drive elements B1, B2 are driven by instrument interface element 601c. Drive interface elements 502a and 502c drive instrument interface elements 601a and 601c respectively. The example calibration rig 700 of FIG. 7 has two rig interface elements 701a and 701b. Rig interface element 701a engages and is driven by drive interface element 502a. Rig interface element 701b engages and is driven by drive interface element 502c. Rig interface element 701b is shaped with respect to drive interface element 502c as described above with reference to rig interface element 701a, drive interface element 502a and instrument interface element 601a. Rig interface element 701b is connected to a damper 702b, which is in turn connected to a force sensor 703b. Damper 702b and force sensor 703b are as described with reference to damper 702a and force sensor 703a. Thus, when attached to the robot arm, the calibration rig 700 engages both the drive interface elements 502a and 502c which drive the opposing end effector elements 409 and 410 to rotate. The calibration rig 700 is thus able to concurrently calibrate both the motors of the robot arm which drive the end effector elements 409, 410 to rotate.

Although not shown in FIG. 7, the calibration rig may have further rig interface elements. For example, the calibration rig may have a third rig interface element to engage with and be driven by drive interface element 502b. Drive interface element 502b drives rotation of the pitch joint 401 of the surgical instrument about the pitch axis 402. This third rig interface element is shaped with respect to drive interface element 502b as described above with reference to rig interface element 701a, drive interface element 502a and instrument interface element 601a. The third rig interface element may be connected to a damper, which is in turn connected to a force sensor. This damper and force sensor are as described with reference to damper 702a and force sensor 703a. Thus, when attached to the robot arm, the calibration rig 700 may engage all of the drive interface elements 502a, 502b and 502c. The calibration rig 700 is thus able to concurrently calibrate the motors of the robot arm which drive the end effector elements 409, 410 to rotate and the motor of the robot arm which drives the instrument to rotate about the pitch axis 402.

In the two examples described above, each rig interface element is connected to its own damper and force sensor. This enables the calibration rig to perform the test sequences described below concurrently on each of the arm motors which drive the drive interface elements to which the rig interface elements are engaged. In an alternative example, two or more of the rig interface elements are connected to the same damper and force sensor of the calibration rig. This enables the calibration rig to be more compact and lighter. However, in this example, the calibration test sequence can only be run on one rig interface element connected to the same damper at once. Hence the calibration test sequence is only run on one of the motors driving a drive interface element engaged with the rig interface elements connected to the same damper at once. So, the calibration test sequence is run on the rig interface elements connected to the same damper and force sensor in succession, rather than concurrently. In this example, as with the examples described above, the calibration rig can remain attached to the robot arm whilst all of the motors of the robot arm are being calibrated.

In a further example, the calibration rig may have only a single rig interface element. That single rig interface element is connected to a damper and force sensor as described in the examples above. That single rig interface element has a complimentary shape and size to each of the drive interface elements. Thus, the single rig interface element can be engaged with and driven by any of the drive interface elements. In this example, the calibration rig is attached to the robot arm with the single rig interface element engaged with a first drive interface element, for example 502a. The calibration test sequence is run on the arm motor driving the first drive interface element. If a further arm motor is to be calibrated, then the calibration rig is detached from the arm, and reattached with the single rig interface element this time engaging a second drive interface element, for example 502c. The calibration test sequence is run on the arm motor driving the second drive interface element. If further arm motors are to be calibrated, then the calibration rig is detached from the arm after each test sequence and reattached with the single rig interface element engaging the drive interface element driven by the next arm motor to be calibrated. The calibration rig of this example is the most compact and light weight, but requires more human interaction than the other examples should more than one motor need to be calibrated.

In the primary example described above, the calibration rig has only two rig interface elements for engaging with the drive interface elements which drive the end effector elements to rotate. This is useful if only the opening and/or closing force of the instrument is to be calibrated, since it enables the calibration rig to be compact and light weight whilst being able to perform the required calibration without needing an operator to detach and reattach the calibration rig.

The damper 702a, 702b may be configured to only provide a resistive force in one direction. In the opposing direction, the damper allows free movement. In the case of a linear damper, the damper only provides a resistive force in one linear direction. In the opposing linear direction, the damper provides free movement. Suitably, the damped motion is provided in the direction which corresponds to the end effector elements 409, 410 being driven to close. In the example of FIG. 7, the damped motion is provided in the direction A, which corresponds to the drive interface elements being driven in a proximal direction from the end of the robot arm towards the base of the robot arm. Correspondingly, the free movement is provided in the direction which corresponds to the end effector elements 409, 410 being driven to open. In the example of FIG. 7, the free movement is provided in the direction B which opposes the direction A. The direction B corresponds to the drive interface elements being driven in a distal direction from the base of the robot arm to the end of the robot arm. The grip performance of the end effector is very important to surgeons, thus calibrating the closing force of the end effector elements is particularly important. If grip performance is the only aspect to be considered, then only providing a resistive force in one direction of the damper (which corresponds to the end effector elements rotating towards each other) enables the grip force of the end effector to be calibrated most efficiently.

Alternatively, the damper 702a, 702b may be configured to provide a resistive force in both opposing directions. This enables the calibration test sequence to be run on the motor when it is driving the drive interface element in both directions. In this case, the rig force sensor 703a is configured to measure the resistive force applied in both directions. This may be implemented using two compression only load cells in-line with the damper, one on either side of the damper. Alternatively, it may be implemented with a single push/pull load cell in-line with the damper which may be positioned on either side of the damper. In the case of the calibration rig of FIG. 7, this would enable the motors driving the end effector elements 409, 410 to rotate to be calibrated for both the closing and opening forces of the end effector elements. Calibrating the motors for opening forces may be useful, for example, for improving the consistency and accuracy of the force applied by the end effector elements during blunt dissection, which is where the surgeon increases the size of a tissue incision by pushing closed end effector elements through the incision and then opening the end effector elements to push the tissue apart.

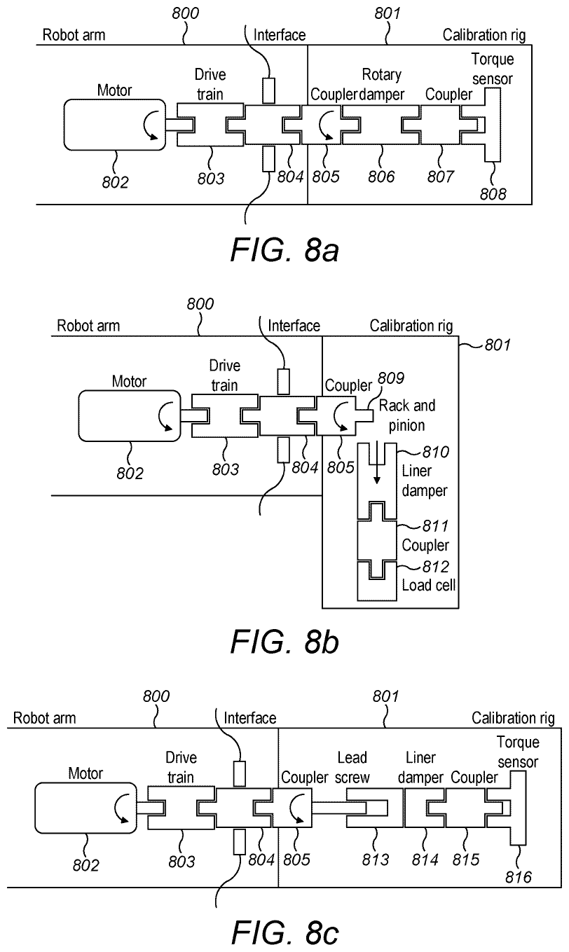

In the examples described above, the calibration rig comprises a linear damper for resisting the motion of rig interface elements which themselves move linearly. The rig interface elements engage drive interface elements which also move linearly. FIGS. 8a, 8b and 8c illustrates examples in which the drive interface elements are configured to provide a rotational drive to the instrument interface elements. The connection between a single motor 802 of the robot arm 800 and the calibration rig 801 is shown in each of FIGS. 8a, 8b and 8c. A drive train 803 connects the robot arm motor 802 to the drive interface element 804. The drive interface element 804 is coupled to the calibration rig 801 via coupler 805. Coupler 805 includes at least a rig interface element.

In FIG. 8a, calibration rig 801 comprises a rotary damper 806 which is attached to coupler 805. Thus, the drive interface element 804 is driven to rotate by motor 802. The drive interface element 804 is engaged with rig interface element of coupler 805 and thereby drives the rig interface element and coupler 805 to rotate. This rotation is coupled to rotary damper 806. Rotary damper 806 provides a resistive force proportional to the angular velocity of the drive interface element 804. The rotary damper 806 is coupled via coupler 807 to a torque sensor 808. Torque sensor 808 measures the resistive force applied by the rotary damper 806. Thus, the calibration rig of FIG. 8a can perform the calibration test sequences described below on the motor 802 of the robot arm utilising the rotary damper 806 as the damper 702a, 702b and the torque sensor 808 in place of the force sensor 703a, 703b. An advantage of utilising a rotary damper 806 is that its stroke/range is not limited by the dimensions of the calibration rig, whereas the stroke/range of a linear damper is limited by its length of travel within the calibration rig.

The apparatus of FIG. 8b differs from that of FIG. 8a in that the calibration rig comprises a linear damper 810 connected to a load cell 812 via a coupler 811. As in FIG. 8a, the drive interface element 804 is engaged with rig interface element of coupler 805 to thereby drive the rig interface element and coupler 805 to rotate. The coupler 805 is coupled to rack and pinion 809. Rack and pinion 809 converts the rotation of the coupler 805 to a linear drive and vice versa. The rack and pinion 809 is connected to the linear damper 810. Thus, the calibration rig of FIG. 8b can perform the calibration test sequences described below on the motor 802 of the robot arm utilising a linear damper 810 and load cell 811 as described above with respect to FIG. 7, and using the rack and pinion to transfer the linear resistive force provided by the linear damper 810 to a force resisting the rotation of the rig interface element and hence the drive interface element 804. This resistive force is measured by the load cell 812.

The apparatus of FIG. 8c also uses a linear damper to provide a resistive force to the rotational drive provided by the robot arm. As in FIG. 8b, a linear damper 814 is connected to a load cell 816 via a coupler 815. As in FIG. 8a, the drive interface element 804 is engaged with rig interface element of coupler 805 to thereby drive the rig interface element and coupler 805 to rotate. The coupler 805 is coupled to lead screw 813. Lead screw 813 converts the rotation of the coupler 805 to a linear drive and vice versa. Lead screw 813 is connected to the linear damper 814. Thus, the calibration rig of FIG. 8c can perform the calibration test sequences described below on the motor 802 of the robot arm utilising a linear damper 814 and load cell 816 as described above with respect to FIG. 7, and using the lead screw to transfer the linear resistive force provided by the linear damper 814 to a force resisting the rotation of the rig interface element and hence the drive interface element 804. This resistive force is measured by the load cell 816.

The calibration rig may comprise a data port for outputting the measurements of the force/torque sensor to an external device. For example, the calibration rig may comprise a USB port for connecting to a control device. The calibration rig may comprise a transmitter for wirelessly transmitting the measurement data to a control device. The calibration rig may comprise a memory for storing the measurement data. Suitably, the calibration rig outputs the measurement data immediately via a USB cable to a control device, thereby not requiring a memory. The calibration rig may be powered through the same USB connection. Alternatively, the calibration rig may be battery powered.

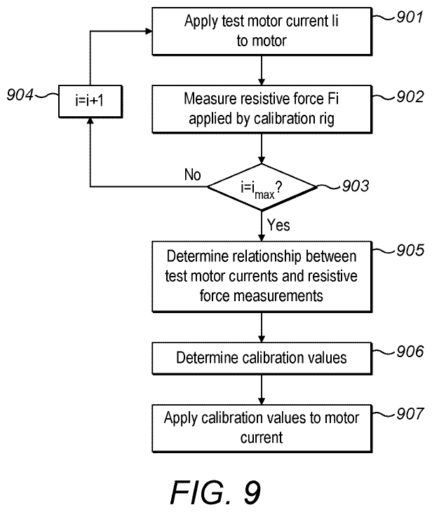

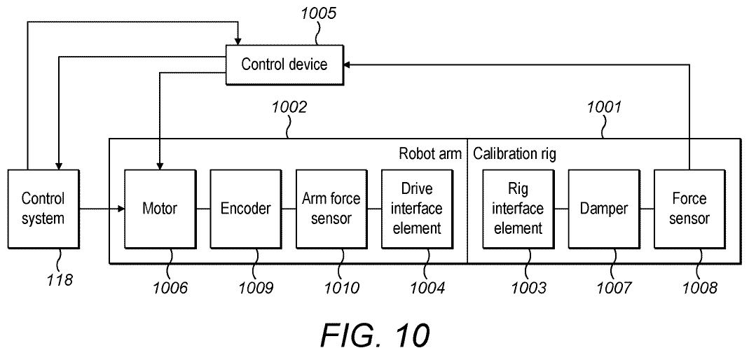

A method of calibrating a motor of the robot arm using a calibration rig as described above will now be described with reference to FIGS. 9 and 10. FIG. 9 is a flowchart illustrating the calibration method. FIG. 10 is a block diagram showing schematically the features of the robotic system relevant to the calibration method.

The calibration rig 1001 is attached to the robot arm 1002. Consequently, each rig interface element 1003 is engaged with a corresponding drive interface element 1004. Suitably, the robot arm 1002 is set up in a predetermined test configuration. The robot arm may be set up into this predetermined test configuration before or after the calibration rig has been attached to the robot arm. The predetermined test configuration is a pose in which either: (i) gravity does not affect the motion of the drive interface element(s), or (ii) gravity affects the motion of the drive interface element(s) in a known way.

A control device 1005 is in communication with the calibration rig 1001 and the robot arm 1002, and the control system 118 described previously. The control device 1005 may be part of the control system 118 or it may be a separate device. For example, the control device 1005 may be a laptop. The control device 1005 controls the calibration method.

The control device 1005 controls a test sequence to be run on the motor 1006, that test sequence comprising a set of motor currents to be applied to the motor 1006. The control device 1005 may directly send control signals to the motor 1006. Alternatively, the control device 1005 may send control signals to the control system 118 which cause the control system 118 to control the motor 1006 to be driven with the set sequence of test motor currents. Thus, a set of test motor currents is applied to the motor 1006. Each test motor current causes the motor 1006 to drive the drive interface element 1004 to move at a constant velocity. As described above, the drive interface element drives the rig interface element 1003. The rig interface element 1003 is connected to damper 1007. The damper 1007 applies a resistive force in response to the rig interface element 1003 being driven. Suitably, that resistive force is proportional to the velocity of the rig interface element, and hence proportional to the velocity of the drive interface element.

At step 901 of the calibration method of FIG. 9, the first test motor current Iᵢ is applied to the motor 1006. At step 902, the resistive force Fᵢ applied by the damper 1007 of the calibration rig 1001 is measured by the force sensor 1008. This measured resistive force Fᵢ is output to the control device 1005. The control device 1005 receives the measured resistive force Fᵢ from the force sensor 1008. The force sensor 1008 may take a plurality of force measurements as the rig interface element 1003 moves. This plurality of force measurements may be averaged to provide a single resistive force to be used in the subsequent calculations. The force sensor 1008 may output all the force measurements to the control device 1005, and the control device 1005 perform the averaging. Alternatively, the force sensor 1008 may comprise logic to average the force measurements it takes for each test motor current, and then output only the averaged force measurement to the control device 1005.

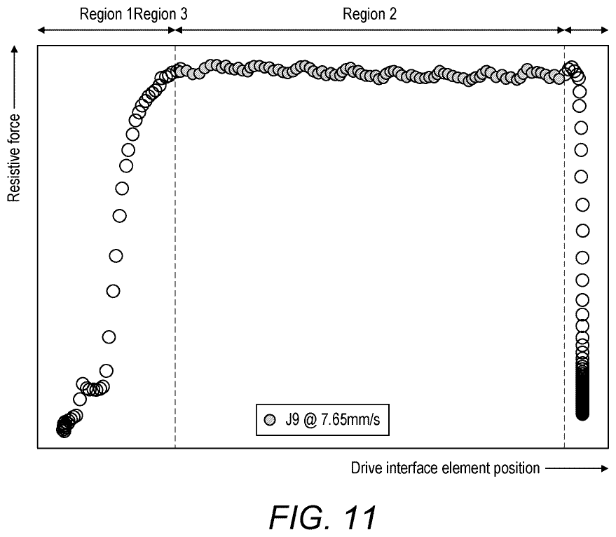

FIG. 11 is a graph illustrating the position of the drive interface element along its displaceable range on the x-axis against the resistive force measured by the force sensor 1008 for a test motor current applied to the motor. The position of the drive interface element along its displaceable range is measured by an encoder 1009 at the output of the motor 1006.

The encoder measures the rotary position of the driveshaft which drives the lead screw which drives the drive interface element to move along its displaceable range. Region 1 is a non-linear region whilst the drive interface element accelerates from stationary to the constant velocity. Region 2 is a linear region during which the drive interface element moves at a constant velocity and the damper responds by applying a constant resistive force. Region 3 is a non-linear region whilst the drive interface element comes to a stop. Suitably, the measurements taken during the linear region 2 are averaged to yield a resistive force measurement for that test motor current. Thus, only the measurements taken during the region 2 of constant resistive force applied by the calibration rig are used in the subsequent calibration process. Different methods may be used to select the data points used in the measurements of region 2. For example, a series of successive measurements may be taken, and the difference in the resistive force measurements of the successive measurements calculated. Once that difference in the resistive force measurements drops below a threshold difference, the most recent resistive force measurement (or an average of the most recent resistive force measurements) is taken as the resistive force measurement for that test motor current. As another example, a series of successive measurements may be taken, and a linear function fitted to groups of successive measurements until the gradient is 0 or smaller than a threshold gradient. The most recent resistive force measurement (or the average of the group of successive measurements that satisfied the threshold gradient) is then taken as the resistive force measurement for that test motor current. The linear region 2 may be selected as follows. The linear region 2 may be selected as encompassing any resistive force measurements that fall within a threshold deviation from an expected resistive force measurement. That expected resistive force measurement is selected to be proportional to the commanded velocity. Resistive force measurements falling outside of the threshold are not used in the subsequent calibration process. Alternatively, or in addition, the linear region 2 may be selected as starting a fixed distance from the starting position of the drive interface element and ending a fixed distance from the ending position of the drive interface element. The start and end of the fixed distance are determined based on the expected distances travelled by the drive interface element during the acceleration and deceleration periods. Alternatively, or in addition, the linear region 2 may be selected based on a mode value of the resistive force measurements.

At step 903, the control device 1005 determines if there is a further test current to be applied, or whether all the test currents of the set have been applied to the motor. If a further test current is to be applied, then method moves to step 904 where the next test current is selected by the control device 1005. The control device then either directly controls the motor 1006, or controls the control system 118 to control the motor 1006 to apply the next test current to the motor. The method then moves to step 901 where the next test current is applied to the motor. The sequence of steps 901 to 904 repeats until all the test currents in the set have been applied to the motor. Alternatively, the control device 1005 may send the complete set of test currents to the control system 118 in one communication, which the control system 118 then applies to the motor in turn.

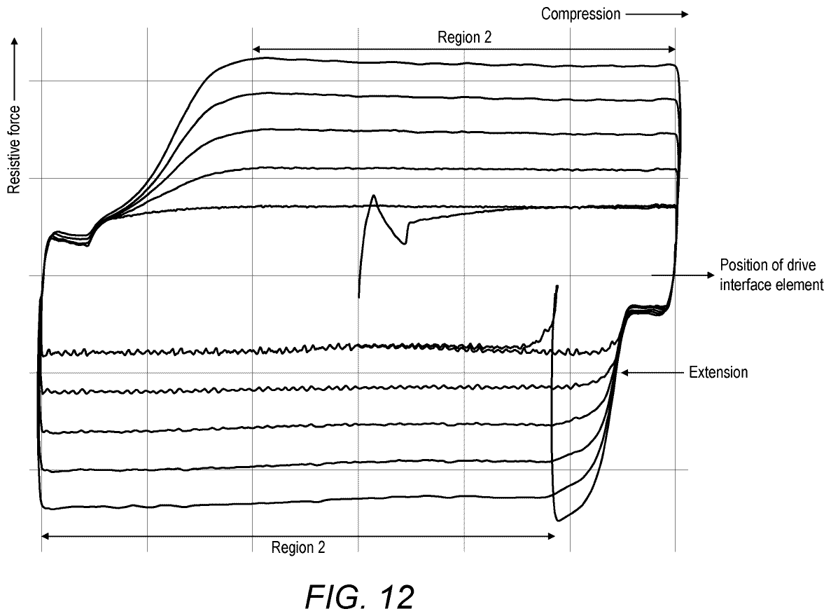

FIG. 12 is a graph illustrating the position of the drive interface element along its displaceable range on the x-axis against the resistive force measured by the force sensor 1008 for each of five test motor currents in a set of test motor currents. Each motor current results in a region 2 in which the drive interface element moves along its travel with constant velocity which results in a constant resistive force measurement. The higher the motor current, the higher the constant velocity and hence the higher the constant resistive force measurement. In this example, the damper is a two-way damper providing a resistive force both when the damper is compressed and when the damper is extended. Thus, the control device 1005 determines an average compression resistive force measurement from the measurements for the drive interface element moving in the compression direction, and separately an average extension resistive force measurement from the measurements for the drive interface element moving in the extension direction. The magnitude of the velocity of the drive interface element may be the same in both the compression and extension directions. In this case, the control device 1005 may determine an average compression resistive force measurement and an average extension resistive force measurement separately as described above. Alternatively, the control device may determine an average resistive force measurement for both the compression and extension directions using the measurements for the drive interface element moving in both the compression and extension directions.

At step 905, the control device determines a relationship between the set of test motor currents applied to motor and the received resistive force measurements. Thus, the control device may determine a relationship between the set of test motor currents and the average resistive force measurement for each test motor current. The control device 1005 may determine a linear relationship between the set of motor currents and the resistive force measurements.

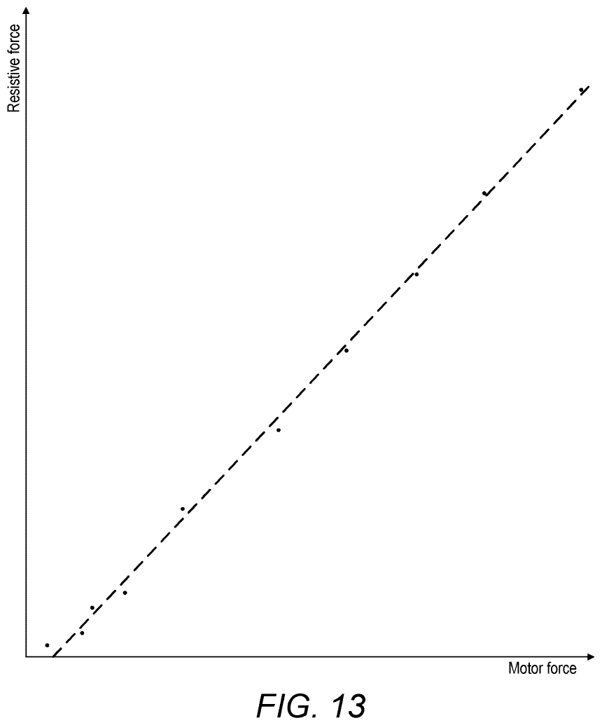

FIG. 13 is a graph illustrating the motor force on the x-axis against the measured resistive force on the y-axis. The motor force is the force the motor is expected to deliver, which is proportional to the current input to the motor. Specifically:

$${τ}_{m} = α {I}_{m} \qquad ( equation 1 )$$

where τₘ is the motor torque, Iₘ is the motor current, and a is a constant.

The current input to the motor is measured using a current loop to ensure that it is the same as the test current commanded by the control device. The measured resistive force applied by the calibration rig is the same as the driving force applied by the drive interface element to the rig interface element. Thus, FIG. 13 illustrates the force expected to be applied by the drive interface element on the x-axis against the force actually applied by the drive interface element on the y-axis. The relationship between the two is linear. A straight line can be fitted to the data, for example using a least-squares method. This straight line has the form:

$$y = mx + c \qquad ( equation 2 )$$

where y is the measured resistive force, and x is the motor force. The gradient m is the efficiency of transferring motor current to driving force of the drive interface element. Typically,

$$m = \frac{joint efficiency}{gear ratio} .$$

The y-intercept is a measure of the joint friction. The y-intercept may be 0.

It will be understood that FIG. 13 is for illustrative purposes. The control device 1005 may determine the force expected to be applied by the drive interface element, however there is no need to. Instead, the control device 1005 may directly determine the relationship between the test motor current and the measured resistive force.

At step 906, the control device 1005 determines calibration values to apply to the motor. The control device uses the determined relationship between the set of test motor currents and the resistive force measurements, along with the known relationship between the resistive force applied by the calibration rig and the driving force applied by the drive interface element to determine the calibration values. Where the determined relationship is linear as described above, and the resistive force applied by the calibration rig is equal to the driving force applied by the drive interface element, then the calibration value(s) is a factor and/or offset.

Where the damper provides resistance both in compression and extension, the control device 1005 may determine a different linear relationship and hence different calibration values for the motor current for the compression and extension.

At step 907, the control device 1005 controls the calibration value(s) to be applied to subsequent motor currents applied to the motor. Suitably, the control device 1005 sends a control signal to the control system 118 to calibrate subsequent motor currents which it causes to be applied to the motor 1006 with the calibration value(s). Thus, during subsequent operation when the calibration rig is detached from the robot arm and an instrument attached to the robot arm, the control system 118 controls the motor 1006 to apply the desired driving forces to an instrument interface element of the instrument by driving the motor with the calibrated motor current.

Further motors of the robot arm may be calibrated by repeating the method described with respect to FIG. 9 to those other motors. The calibration method may be carried out concurrently on the motors if the calibration rig has a rig interface element, damper and force sensor for each drive interface element. Alternatively, the calibration method may be carried out in sequence on the motors.

The robot arm may comprise an arm force sensor configured to measure the driving force applied by a motor to a drive interface element. The robot arm may comprise a set of arm force sensors which measure the forces acting on a set of drive interface elements. The robot arm may comprise a set of arm force sensors, each configured to measure the driving force applied by a respective motor to a respective drive interface element. Alternatively, the robot arm may comprise a single arm force sensor which measures the forces acting on each of a set of drive interface elements.

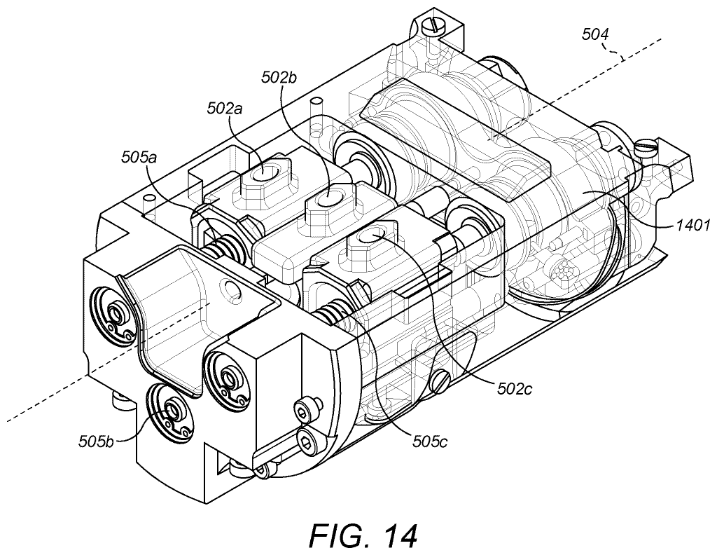

FIG. 14 illustrates the drive assembly interface as shown in FIG. 5, but additionally shows a force sensor 1401. This force sensor has a set of load cells, each load cell for measuring the driving force applied to one of the drive interface elements 502a, 502b, 502c. These measurements can be used to deduce the tension forces in the driving elements of the instrument. For each drive interface element, the load cell for that drive interface element measures the axial force exerted by the lead screw 505a, 505b, 505c when that lead screw is rotating in order to move the drive interface element. The force sensor of FIG. 14 is a unitary component with three load cells. The force sensor is fixed to the chassis of the drive assembly and sandwiched between thrust bearings. Each load cell surrounds the drive shaft which connects the motor to the lead screw for a drive interface element. The drive shaft is free to rotate inside the load cell. As the drive shaft rotates to move the drive interface element towards the end of the robot arm, the drive interface element exerts an opposite force against the lead screw which is transferred to the side of the load cell which faces the distal end of the robot arm. As the drive shaft rotates in the other rotational direction to move the drive interface element towards the base of the robot arm, the drive interface element exerts an opposite force against the lead screw which is transferred to the side of the load cell which faces the bulk of the robot arm. Thus, the load cell measures the driving force applied to the drive interface element when it is driven both towards and away from the instrument.

The load cell measures a voltage, and converts this to a force measurement. The measured voltage is proportional to the force measurement.

$${F}_{m} = {βV}_{m} \qquad ( equation 3 )$$

where Fₘ is the force measurement, Vₘ is the measured voltage, and β is a constant. β accounts for the rated sensitivity and capacity of the strain gauge of the load cell, and also a stimulus voltage.

The arm force sensor 1401 may suffer from thermal drift. It exhibits a temperature dependence which can lead to it becoming uncalibrated with use. For example, the arm force sensor may suffer from thermal expansion which causes its strain gauge to elongate with temperature, which in turn changes the relationship between the current it measures and the force that corresponds to. The calibration rig described herein can be used to calibrate the arm force sensor 1401.

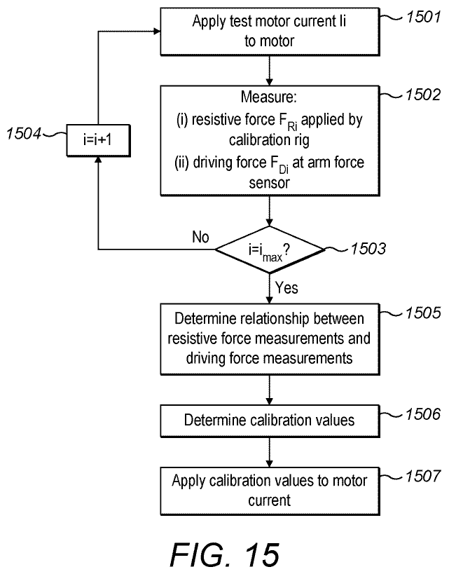

A method of calibrating a force sensor 1010 of the robot arm which measures the driving force applied by motor 1006 to drive interface element 1004 will now be described with reference to FIGS. 15 and 10. The calibration method uses the calibration rig described herein.

A set of test motor currents is applied to the motor 1006. At step 1501 of the calibration method of FIG. 15, the first test motor current Iᵢ is applied to the motor 1006. This is the same as step 901 of FIG. 9. At step 1502, the resistive force F_{Ri} applied by the damper 1007 of the calibration rig 1001 is measured by the force sensor 1008. This measured resistive force F_{Ri} is output to the control device 1005. The control device 1005 receives the measured resistive force F_{Ri} from the force sensor 1008. The force sensor 1008 may take a plurality of force measurements as the rig interface element 1003 moves. This plurality of force measurements may be averaged to provide a single resistive force to be used in the subsequent calculations. The force sensor 1008 may output all the force measurements to the control device 1005, and the control device 1005 perform the averaging. Alternatively, the force sensor 1008 may comprise logic to average the force measurements it takes for each test motor current, and then output only the averaged force measurement to the control device 1005.

At step 1502, the driving force F_{D}i is measured by the arm force sensor 1010. The arm force sensor 1010 outputs this measured driving force F_{D}i to the control device 1005. Alternatively, the arm force sensor 1010 outputs this measured driving force F_{D}i to the control system 118 which then outputs the measured driving force F_{D}i to the control device 1005. The control device 1005 receives the measured driving force F_{D}i. The arm force sensor 1010 may take a plurality of driving force measurements as the rig interface element 1003 moves. This plurality of driving force measurements may be averaged to provide a single driving force measurement to be used in the subsequent calculations. The arm force sensor 1010 may output all the force measurements directly or indirectly to the control device 1005, and the control device 1005 perform the averaging. Alternatively, the arm force sensor 1010 may comprise logic to average the driving force measurements it takes for each test motor current, and then output only the averaged driving force measurement either directly or indirectly to the control device 1005.

At step 1503, the control device 1005 determines if there is a further test current to be applied, or whether all the test currents of the set have been applied to the motor. If a further test current is to be applied, then method moves to step 1504 where the next test current is selected by the control device 1005. The control device then either directly controls the motor 1006, or controls the control system 118 to control the motor 1006 to apply the next test current to the motor. The method then moves to step 1501 where the next test current is applied to the motor. The sequence of steps 1501 to 1504 repeats until all the test currents in the set have been applied to the motor. Alternatively, the control device 1005 may send the complete set of test currents to the control system 118 in one communication, which the control system 118 then applies to the motor in turn.

At step 1505, the control device determines a relationship between the received resistive force measurements and the received driving force measurements. Thus, the control device may determine a relationship between the average resistive force measurements and the average driving force measurements. The control device 1005 may determine a linear relationship between the resistive force measurements and the driving force measurements.

At step 1506, the control device 1005 determines calibration values to apply to the arm force sensor 1010. The control device uses the determined relationship between the resistive force measurements and the driving force measurements to determine the calibration values. Where the determined relationship is linear as described above, the calibration value(s) is a factor and/or offset.

Where the damper provides resistance both in compression and extension, the control device 1005 may determine a different linear relationship and hence different calibration values for the arm force sensor 1010 for the compression and extension.

At step 1507, the control device 1005 controls the calibration value(s) to be applied to the arm force sensor 1010. Suitably, the control device 1005 sends a control signal to the control system 118 to calibrate subsequent driving force measurements of the arm force sensor 1010 with the calibration value(s). Thus, during subsequent operation when the calibration rig is detached from the robot arm and an instrument attached to the robot arm, the arm force sensor 1010 driving force measurements are calibrated.

Further arm force sensors of the robot arm may be calibrated by repeating the method described with respect to FIG. 15 for the motor and drive interface element connected to each arm force sensor to be calibrated. The calibration method may be carried out concurrently on the arm force sensors if the calibration rig has a rig interface element, damper and force sensor for each drive interface element. Alternatively, the calibration method may be carried out in sequence on the arm force sensors.

The calibration method described with respect to FIG. 15 for calibrating the arm force sensor may be performed separately to, or alongside, the calibration method described with respect to FIG. 9 for calibrating the arm motor. If the method of FIG. 15 is performed alongside the method of FIG. 9, then the test sequence comprising the set of test motor currents is applied to the motor once. For each test motor current, both: (i) the resistive force measurements applied by the calibration rig are measured as discussed in step 902 of FIG. 9 and step 1502 of FIG. 15; and (ii) the driving force measurements of the arm force sensor are measured as discussed in step 1502 of FIG. 15. Both a relationship between the test motor currents and the resistive force measurements, and a relationship between the resistive force measurements and the driving force measurements are determined by the control device 1005 as described with reference to steps 905 and 1505. Calibration values for both the motor and arm force sensor are determined by the control device as described with reference to steps 906 and 1506. These calibration values are subsequently applied to both the motor and arm force sensor as described with reference to steps 907 and 1507.

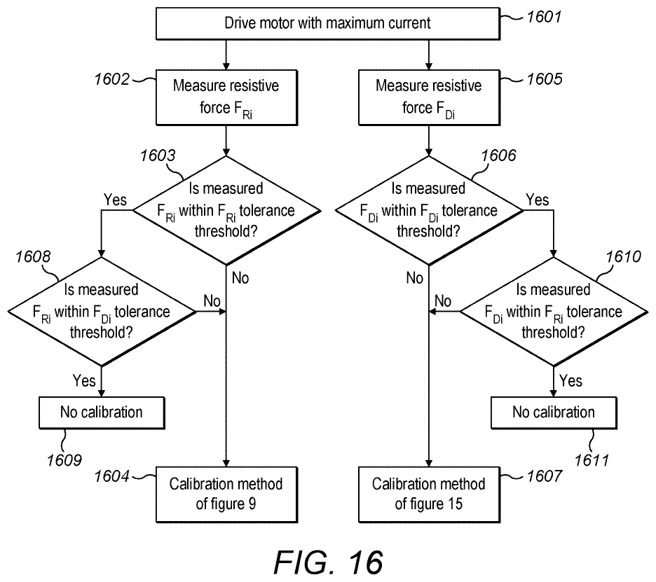

The calibration methods described with reference to FIGS. 9 and 15 may be performed in response to determining that certain tolerances are not met. FIG. 16 is a flowchart illustrating a method which may be performed when the calibration rig is attached to the robot arm to determine whether the motor and/or arm force sensor coupled to a drive interface element is/are to be calibrated.

At step 1601 of FIG. 16, the control device 1005 controls the motor 1006 to be driven with maximum current. Whilst the motor is being driven with maximum current, the calibration rig measures the resistive force applied by the damper at step 1602, and the arm force sensor measures the driving force of the drive interface element at step 1605. The control device 1005 receives these measurements via the routes described with reference to FIGS. 9 and 15. At step 1603, the control device compares the measured resistive force to a resistive force tolerance threshold. If the measured resistive force is not within the resistive force tolerance threshold, then the control device moves to step 1604 where it implements the calibration method of FIG. 9. At step 1606, the control device compares the measured driving force to a driving force tolerance threshold. If the measured driving force is not within the driving force tolerance threshold, then the control device moves to step 1607 where it implements the calibration method of FIG. 15.

If, at step 1603, the control device determines that the measured resistive force is within the resistive force tolerance threshold, then it may go on to determine, at step 1608, whether the measured resistive force is within the driving force tolerance threshold. If the measured resistive force is not within the driving force tolerance threshold, then the control device moves to step 1604 where it implements the calibration method of FIG. 9. If the measured resistive force is within the driving force tolerance threshold, then the control device moves to step 1609 where it determines not to perform a calibration of the motor.

If, at step 1606, the control device determines that the measured driving force is within the driving force tolerance threshold, then it may go on to determine, at step 1610, whether the measured driving force is within the resistive force tolerance threshold. If the measured driving force is not within the resistive force tolerance threshold, then the control device moves to step 1607 where it implements the calibration method of FIG. 15. If the measured driving force is within the resistive force tolerance threshold, then the control device moves to step 1611 where it determines not to perform a calibration of the arm force sensor.

Whilst the test sequence is being run, the control device may determine to stop the test if any one or more of the following conditions are met:
1. The measured resistive force exceeds a predetermined force limit. This predetermined force limit is higher than any anticipated driving forces during the calibration process. The control device compares the measured resistive forces it receives to the predetermined force limit. It stops the test if the predetermined force limit is exceeded in order to protect the drive train from excessive forces.
2. The joint is delivering its maximum available force. The control device compares the measured resistive forces it receives to the maximum joint force. It stops the test if this maximum joint force is met because the force provided is unstable and thus not suitable for use in the calibration process.
3. The measured resistive force is lower than a predetermined threshold. The control device compares the measured resistive forces it receives to the predetermined threshold. It stops the test if the measured resistive force is lower than the predetermined threshold. This can happen if the damper in the calibration rig has a low damping rate meaning that excessive velocity is required to achieve the forces that the control device is expecting to test from the applied motor currents. This condition is to protect the drive system from driving at excessive speeds.

Having determined to stop the test, the control device sends a control signal to the control system 118. The control system 118 responds to this control signal by stopping application of the test motor current to the motor.

The motor currents in the set of test motor currents may be chosen using speed iteration. In other words, the difference between the motor currents of successive iterations of the test may reduce as the motor current increases towards a maximum current. That maximum current may correspond to a target force. The target force may be a peak force. The peak force is important for the safe functioning of the robot arm, thus more measurements are taken around that peak force thereby improving the confidence in those measurements.

The control device 1005 may, for each test motor current, determine the velocity of the drive interface element. Specifically, the control device may determine the mean velocity of the drive interface element in the linear region 2 shown in FIG. 11. The control device receives the position of the drive interface element along its displaceable range from the encoder 1009, and the time that the drive interface element is at that position. Thus, the control device determines a velocity of the drive interface element between successive encoder readings. An average of the velocity readings determined in linear region 2 of FIG. 11 provides a mean velocity of the drive interface element. This mean velocity is used as the measured constant velocity of the drive interface element.

The control device 1005 may, for each test motor current, measure the distance travelled by the drive interface element whilst the drive interface element moves at constant velocity. The distance travelled is determined from the difference between the first and last encoder readings taken during the linear region 2 of FIG. 11, i.e. during the period of time that the drive interface element is determined to have moved at constant velocity.

Suitably, following either or both of steps 907 and 1507 in which the calibration values have been applied to the motor current, the control device 1005 causes a verification process to be run.

To verify the motor calibration, the control device firstly applies a verification motor current to the motor to drive the drive interface element to move at a verification velocity. For that verification motor current, the verification resistive force applied by the calibration rig is measured. That measured verification resistive force is then compared to a target force. If the verification resistive force is within a threshold of the target force, the verification of the motor calibration is determined to be successful. If the verification resistive force is outside the threshold T₁ of the target force, then the calibration process of FIG. 9 is repeated. The threshold T₁ may, for example, be a value such that T₁<10 N, or T₁<5 N. Suitably, the verification motor current is a maximum motor current, thereby causing the drive interface element to move at its peak velocity providing a peak force.

To verify the arm force sensor calibration, the control device firstly applies a verification motor current to the motor to drive the drive interface element to move at a verification velocity. For that verification motor current, the verification resistive force applied by the calibration rig is measured and the verification driving force at the arm force sensor is measured. The measured verification resistive force applied by the calibration rig is compared to the verification driving force of the arm force sensor. If the verification driving force is within a threshold T₂ of the verification resistive force, then the verification of the arm force sensor calibration is determined to be successful. If the verification driving force is outside the threshold T₂ of the verification resistive force, then the calibration process of FIG. 15 is repeated. The threshold T₂ may, for example, be a value such that T₂<10N, or T₂<5N. Suitably, the verification motor current is a maximum motor current, thereby causing the drive interface element to move at its peak velocity providing a peak force.

Both the verification of the motor calibration and the verification of the arm force sensor calibration may be carried out concurrently by driving the motor with the same verification motor current for each verification process, and using the measured verification resistive force for both verifications.

The examples described herein enable the current applied to each motor of the robot arm to be calibrated in a time efficient manner which does not require an operator to be present throughout the calibration process. The described calibration rig is also compact enabling it to be used as a portable tool by service engineers.

Calibration of the motors which drive the drive interface elements is particularly important because those drive interface elements control the grip performance of the surgical instrument and hence the life of the instrument. The efficiency of the performance of the drive interface elements' transfer of force to the instrument interface elements changes over time, thus regular calibration of them is important. By correcting for the thermal drift experienced by the arm force sensors, the use of their measurements in further control algorithms and telemetry analysis is enabled.

In the examples described above, only measurements taken during the linear region 2 shown in FIG. 11 are used in the calibration process. Alternatively, measurements taken during the non-linear regions 1 and 3 could be used in the calibration process in addition to those measurements taken during the non-linear region 2 if the non-linear relationships of regions 1 and 3 are known. In this case, the backlash which occurs particularly in region 1 when initially applying current to the motor to drive the drive interface element would need to be modelled.
In the examples described above, during the test sequence, the motor is driven at constant velocity. This results in the linear region 2 shown in FIG. 11. Alternatively, the motor may be driven at a non-constant velocity. A relationship between motor current and the measured resistive force applied by the damper could still be determined.

In the examples described above, a force sensor in the calibration rig measures the resistive force applied by the damper of the calibration rig. This force sensor could be omitted if the relationship between the force exerted by the damper and the velocity of the drive interface element is well modelled, and if the test is carried out in a constant temperature environment. Heating a hydraulic damper reduces the resistive forces at the same velocities due to a reduction in the viscosity of the fluid at higher temperatures. Thus, where the temperature of the environment is not consistent and a hydraulic damper is used, a force sensor is also used. In this case where the temperature is consistent and the relationship is well modelled, the relationship between the input motor current and the measured velocity of the drive interface element is determined, using measurements from the position encoder 1009. The known relationship between the force exerted by the damper and the velocity of the drive interface element may be used to convert this determined relationship to a relationship between the input motor current and the resistive force of the damper. From this, calibration values for the motor current may be determined as previously described. Alternatively, the determined relationship between the input motor current and the measured velocity of the drive interface element may be used to directly determine the calibration values for the motor current.

In the examples described herein, the damper is a passive component, for example a hydraulic damper. However, the damper could alternatively be an active component which is driven by a motor. A passive component different to a damper may be used to provide a resistive force against movement of the rig interface element, and hence against movement of the drive interface element. For example, a spring may be used to passively resist motion of the rig interface element.

The applicant hereby discloses in isolation each individual feature described herein and any combination of two or more such features, to the extent that such features or combinations are capable of being carried out based on the present specification as a whole in the light of the common general knowledge of a person skilled in the art, irrespective of whether such features or combinations of features solve any problems disclosed herein, and without limitation to the scope of the claims. The applicant indicates that aspects of the present invention may consist of any such individual feature or combination of features. In view of the foregoing description it will be evident to a person skilled in the art that various modifications may be made within the scope of the invention.

1. A method configured to calibrate a motor of a surgical robot arm using a calibration rig, the motor configured to drive a drive interface element of the surgical robot arm, the drive interface element configured to drive an instrument interface element of a surgical instrument attached to the surgical robot arm to thereby drive a distal end effector of the surgical instrument, the calibration rig comprising a rig interface element which engages with and is driven by the drive interface element, the method comprising:
running a test sequence comprising:
controlling a set of test motor currents to be applied to the motor, each test motor current causing the motor to drive the drive interface element to move; and
for each test motor current of the set of test motor currents, receiving a measured resistive force applied by a calibration rig, the calibration rig applying the resistive force in response to the rig interface element being driven by the drive interface element;
determining a relationship between the set of test motor currents and the resistive force measurements;
determining calibration value(s) from: (i) the determined relationship, and (ii) a known relationship between the resistive force applied by the calibration rig and the driving force applied by the drive interface element; and
controlling the calibration value(s) to be applied to subsequent motor currents applied to the motor so as to cause the motor to drive the drive interface element to apply desired driving forces to an instrument interface element of an attached surgical instrument.

2. (canceled)

3. The method of claim 1, further comprising, for each test motor current measuring the resistive force applied by the calibration rig at the calibration rig.

4. The method of claim 1, wherein the resistive force applied by the calibration rig is proportional to the velocity of the drive interface element, and the determined relationship is a linear relationship, the calibration value(s) being a factor and/or offset.

5. (canceled)

6. The method of claim 1, wherein the resistive force applied by the calibration rig is the same as the driving force applied by the drive interface element.

7. (canceled)

8. (canceled)

9. (canceled)

10. The method of claim 1, further comprising setting up the robot arm in a predetermined test configuration prior to running the test sequence.

11. The method of claim 1, wherein the surgical robot arm comprises a further motor configured to drive a further drive interface element, the further drive interface element configured to drive a further instrument interface element of the surgical instrument, the method further comprising repeating the steps of claim 1 to calibrate the further motor.

12. The method of claim 11, comprising implementing the steps of claim 1 for the motor and the further motor concurrently.

13. The method of claim 1, wherein the surgical robot arm comprises an arm force sensor configured to measure the driving force applied by the motor to the drive interface element, wherein the method further comprises:
for each test motor current of the set of test motor currents, measuring the driving force at the arm force sensor;
determining a further relationship between the set of test motor currents and the driving force measurements;
determining further calibration value(s) from the determined further relationship; and
applying the calibration value(s) to the arm force sensor,
wherein the determined further relationship is a linear relationship, the calibration value(s) being a factor and/or offset.

14. (canceled)

15. The method of claim 13, further comprising:
driving the motor with a maximum current;
whilst driving the motor with the maximum current:
measuring the resistive force, and
at the arm force sensor, measuring the driving force applied by the motor to the drive interface element;
comparing the measured resistive force to a resistive force tolerance threshold;
comparing the measured driving force to a driving force tolerance threshold; and
if either the measured resistive force does not meet the resistive force tolerance threshold, or the measured driving force does not meet the driving force tolerance threshold, performing the calibration method of claim 1.

16. The method of claim 15, further comprising:
comparing the measured resistive force to the driving force tolerance threshold; and
if the measured resistive force does not meet the driving force tolerance threshold, performing the calibration method of claim 1.

17. The method of claim 16, further comprising:
comparing the measured driving force to the resistive force tolerance threshold; and
if the measured driving force does not meet the resistive force tolerance threshold, performing the calibration method of claim 1.

18. The method of claim 1, further comprising:
comparing the measured resistive force to a predetermined force limit, and
halting the test sequence if the measured resistive force exceeds the predetermined force limit.

19. The method of claim 1, further comprising, for each test motor current of the set of test motor currents, measuring a constant velocity of the drive interface element; and
the distance travelled by the drive interface element whilst the drive interface element moves at a constant velocity.

20. (canceled)

21. The method of claim 1, further comprising verifying the motor calibration by:
applying a verification motor current to the motor to drive the drive interface element to move at a verification velocity;
for that verification motor current, measuring the verification resistive force applied by the calibration rig; and
comparing the measured verification resistive force to a target force.

22. The method of claim 21, further comprising verifying the arm force sensor calibration by:
for the verification motor current, measuring the verification driving force at the arm force sensor; and
comparing the measured verification resistive force to the verification driving force.

23. A method of calibrating an arm force sensor of a surgical robot arm using a calibration rig, the arm force sensor configured to measure the driving force applied by a motor of the robot arm to a drive interface element of the robot arm, the drive interface element configured to drive an instrument interface element of a surgical instrument attached to the surgical robot arm to thereby drive a distal end effector of the surgical instrument, the calibration rig comprising a rig interface element which engages with and is driven by the drive interface element, the method comprising:
running a test sequence comprising:
controlling a set of test motor currents to be applied to the motor, each test motor current causing the motor to drive the drive interface element to move; and
for each test motor current of the set of test motor currents, receiving (i) a measured resistive force applied by the calibration rig, the calibration rig applying the resistive force in response to the rig interface element being driven by the drive interface element, and (ii) a measured driving force at the arm force sensor;
determining a relationship between the resistive force measurements and the driving force measurements;
determining calibration value(s) from the determined relationship; and
controlling the calibration value(s) to be applied to the arm force sensor.

24. A calibration rig configured to calibrate a motor of a surgical robot arm, the motor configured to drive a drive interface element of the surgical robot arm, the drive interface element configured to drive an instrument interface element of a surgical instrument attached to the surgical robot arm to thereby drive a distal end effector of the surgical instrument, the calibration rig comprising:
a rig interface element shaped so as to engage with and be driven by the drive interface element;
a damper configured to provide a resistive force in response to the rig interface element being driven by the drive interface element; and
a rig force sensor configured to measure the resistive force applied by the damper, for each of a set of test motor currents that is applied to the motor to drive the drive interface element to move.

25. A calibration rig as claimed in claim 24, wherein the damper is a linear damper configured to provide a resistive force proportional to the constant velocity of the driven drive interface element,
wherein the linear damper is configured to only provide the resistive force in one linear direction.

26. (canceled)

27. A calibration rig as claimed in claim 2524, wherein the damper is a linear damper configured to provide a resistive force proportional to the constant velocity of the driven drive interface element, wherein the linear damper is configured to provide the resistive force in two opposing linear directions.

28. (canceled)

29. A calibration rig as claimed in claim 24, further comprising a further rig interface element shaped so as to engage with and be driven by a further drive interface element;
a further damper configured to provide a resistive force in response to the further rig interface element being driven by the further drive interface element; and
a further rig force sensor configured to measure the resistive force applied by the further damper.

30. (canceled)

31. (canceled)
